(19) United States
(12) Patent Application Publication
(10) Pub. No.: US 2007/0149778 A1
(43) Pub. Date: Jun. 28, 2007

(54) BECKMANN REARRANGEMENT USING MOLECULAR SIEVE SSZ-74

(75) Inventors: Stacey I. Zones, San Francisco, CA (US); Allen W. Burton JR., Richmond, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 11/614,726

(22) Filed: Dec. 21, 2006

Related U.S. Application Data
(60) Provisional application No. 60/754,867, filed on Dec. 28, 2005.

(51) Int. Cl. C07D 201/04

(52) U.S. Cl. 540/536

(57) ABSTRACT

The present invention relates to new crystalline molecular sieve SSZ-74 prepared using a hexamethylene-1,6-bis-(N-methyl-N-pyrrolidinium) dication as a structure-directing agent, and its use in catalysts for Beckmann rearrangement.

This application claims the benefit under 35 USC 119 of Provisional Application No. 60/754,867, filed Dec. 28, 2005.

### BACKGROUND OF THE INVENTION

1. Field of the Invention

The present invention relates to the use of new crystalline molecular sieve SSZ-74 in catalysts in Beckmann rearrangement reactions.

2. State of the Art

Because of their unique sieving characteristics as well as their catalytic properties, crystalline molecular sieves and zeolites are especially useful in applications such as hydrocarbon conversion, gas drying and separation. Although many different crystalline molecular sieves have been disclosed, there is a continuing need for new molecular sieves with desirable properties for gas separation and drying, hydrocarbon and chemical conversions, and other applications. New molecular sieves may contain novel internal pore architectures, providing enhanced selectivities in these processes.

### SUMMARY OF THE INVENTION

The present invention is directed to a family of crystalline molecular sieves with unique properties, referred to herein as “molecular sieve SSZ-744” or simply “SSZ-74”.

In accordance with the present invention there is provided a process for the preparation of amides from oximes via Beckmann rearrangement comprising contacting the oxime in the vapor phase with a catalyst comprising a crystalline molecular sieve having a mote ratio greater than about 15 of (1) an oxide of a first tetravalent element to (2) an oxide of a trivalent element, pentavalent element, second tetravalent element which is different from said first tetravalent element or mixture thereof and having, after calcination, the X-ray diffraction lines of Table II. It should be noted that the phrase “mole ratio greater than about 15” includes the case where there is no oxide (2), i.e., the mole ratio of oxide (1) to oxide (2) is infinity. In that case the molecular sieve is comprised of essentially all silicon oxide. Preferably, the molecular sieve is acidic.

The present invention also provides such a process wherein the crystalline molecular sieve has a mole ratio greater than about 15 of (1) silicon oxide to (2) an oxide, selected from aluminum oxide, gallium oxide, iron oxide, boron oxide, titanium oxide, indium oxide and mixtures thereof, and having, after calcination, the X-ray diffraction lines of Table II.

### BRIEF DESCRIPTION OF THE DRAWING

FIG. 1 shows a comparison of two X-ray diffraction patterns, the top one being ZSM-5 and the bottom one being SSZ-74.

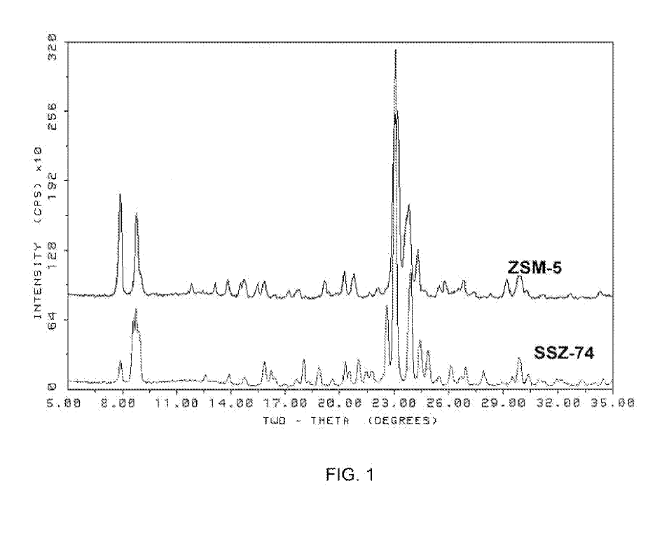

### DETAILED DESCRIPTION OF THE INVENTION

The present invention relates to a molecular sieve designated herein “molecular sieve SSZ-74” or simply “SSZ-74”.

The present invention relates to a process for the preparation of amides from oximes The present invention further relates to the use of SSZ-74 in the catalytic transformation of oximes, such as cyclohexanone oxime, to amides, such as epsilon-caprolactam (caprolactam), also known as Beckmann catalytic rearrangement. The Beckmann rearrangement is shown below (where sulfuric acid is used instead of a molecular sieve catalyst).

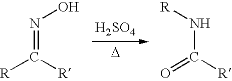

Amides, and in particular caprolactam, are known in literature as important intermediates for chemical syntheses and as raw materials for the preparation of polyamide resins.

Caprolactam is produced industrially by cyclohexanone oxime rearrangement in liquid phase using sulfuric acid or oleum. The rearranged product is neutralized with ammonia causing the joint formation of ammonium sulfate. This technology has numerous problems linked to the use of sulfuric acid, to the formation of high quantities of ammonium sulfate, with relative problems of disposal, corrosion of the equipment owing to the presence of acid vapors, etc.

Alternative processes have been proposed in the literature for the catalytic rearrangement of cyclohexanone oxime into caprolactam, in which solids of an acid nature are used, as catalysts, selected from derivatives of boric acid, zeolites, non-zeolitic molecular sieves, solid phosphoric acid, mixed metal oxides, etc.

In particular, European patent 234.088 describes a method for preparing caprolactarm which comprises putting cyclohexanone oxime in gaseous state in contact with alumino-silicates of the zeolitic type such as ZSM-5, ZSM-11 or ZSM-23 having a “Constraint Index” of between 1 and 12, an atomic ratio Si/Al of at least 500 (SiO₂/Al₂O₃ mote ratio of at least 1,000) and an external acid functionality of less than 5 micro equivalents/g.

Zeolites, as described in “Zeolite Molecular Sieves” D. W. Breck, John Wiley & Sons, (1974) or in “Nature” 381 (1996), 295, are crystalline products characterized by the presence of a regular microporosity, with channels having dimensions of between 3 and 10 Angstroms. In some particular zeolitic structures there can be cavities with greater dimensions, of up to about 13 Angstroms.

With the aim of providing another method for the preparation of amides, and in particular of caprolactam, a new process has now been found which uses a catalyst comprising SSZ-74. The present invention therefore relates to a process for the preparation of amides via the catalytic rearrangement of oximes which comprises putting an oxime in vapor phase in contact with a catalyst comprising a crystalline molecular sieve having a mole ratio greater than about 15 of (1) an oxide of a first tetravalent element to (2) an oxide of a trivalent element, pentavalent element, second tetravalent element which is different from said first tetravalent element or mixture thereof and having, after calcination, the X-ray diffraction lines of Table II. The molecular sieve may have a mole ratio greater than about 15 of (1) silicon oxide to (2) an oxide selected from aluminum oxide, gallium oxide, iron oxide, boron oxide, titanium oxide, indium oxide and mixtures thereof.

Other methods for converting oximes to amides via Beckmann rearrangement are disclosed in U.S. Pat. No. 4,883,915, issued Nov. 28, 1989 to McMahon, which uses a crystalline borosilicate molecular sieve in the catalyst and U.S. Pat. No. 5,942,613, issued Aug. 24, 1999 to Carati et al., which uses a mesoporous silica-alumina in the catalyst. Both patents are incorporated by reference herein in their entirety.

According to the present invention the preferred amide is epsilon-caprolactam (caprolactam) and the preferred oxime is cyclohexanone oxime (CEOX). In particular, the catalytic rearrangement of the cyclohexanone oxime takes place at a pressure of between 0.05 and 10 bars and at a temperature of between 250° C. and 500° C., preferably between 300° C. and 450° C. More specifically, the cyclohexanone oxime, in vapor phase, is fed to the reactor containing the catalyst in the presence of a solvent and optionally an incondensable gas. The cyclohexanone oxime is dissolved in the solvent and the mixture thus obtained is then vaporized and fed to the reactor. The solvent should be essentially inert to the oxime and the amide, as well as the catalyst. Useful solvents include, but are not limited to, lower boiling hydrocarbons, alcohols and ethers.

Preferred solvents are of the type R¹—O—R² wherein R¹ is a C₁-C₄ alkyl chain and R² can be a hydrogen atom or an alkyl chain containing a number of carbon atoms less than or equal to R¹. These solvents can be used alone or mixed with each other or combined with an aromatic hydrocarbon such as benzene or toluene. Alcohols with a C₁-C₂ alkyl chain are particularly preferred.

The cyclohexanone oxime is fed to the rearrangement reactor with a weight ratio with respect to the catalyst which is such as to give a WHSV (Weight Hourly Space Velocity), expressed as Kg of cyclohexanone oxime/kg of catalyst/time, of between 0.1 and 50 hr.⁻¹, preferably between 0.5 and 20 hr.⁻¹.

The deterioration of the catalyst is due to the formation of organic residues which obstruct the pores of the catalyst and poison its active sites. The deterioration process is slow and depends on the operating conditions and in particular the space velocity, solvent, temperature, composition of the feeding. The catalytic activity however can be efficiently reintegrated by the combustion of the residues, by treatment in a stream of air and nitrogen at a temperature of between 450° C. and 600° C.

In preparing SSZ-74, a hexamethylene-1,6-bis-(N-methyl-N-pyrrolidinium) dication is used as a structure directing agent (“SDA”), also known as a crystallization template. The SDA useful for making SSZ-74 has the following structure:

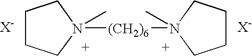

Hexamethylene-1,6-bis-(N-methyl-N-pyrrolidinium) dication

The SDA dication is associated with anions (X⁻) which may be any anion that is not detrimental to the formation of the SSZ-74. Representative anions include halogen, e.g., fluoride, chloride, bromide and iodide, hydroxide, acetate, sulfate, tetrafluoroborate, carboxylate, and the like. Hydroxide is the most preferred anion. The structure directing agent (SDA) may be used to provide hydroxide ion. Thus, it is beneficial to ion exchange for example, a halide to hydroxide ion.

In general, SSZ-74 is prepared by contacting (1) an active source(s) of silicon oxide, and, optionally, (2) an active source(s) of aluminum oxide, gallium oxide, iron oxide boron oxide, titanium oxide, indium oxide and mixtures thereof with the hexamethylene 1,6-bis-(N-methyl-N-pyrrolidinium) dication SDA in the presence of fluoride ion.

SSZ-74 is prepared from a reaction mixture comprising, in terms of mole ratios, the following:

TABLE A
Reaction Mixture

| | Typical | Preferred |
|---|---|---|
| SiO₂/XₐO_{b} | 100 and greater | |
| OH—/SiO₂ | 0.20-0.80 | 0.40-0.60 |
| Q/SiO₂ | 0.20-0.80 | 0.40-0.60 |
| M_{2/n}/SiO₂ | 0-0.04 | 0-0.025 |
| H₂O/SiO₂ | 2-10 | 3-7 |
| HF/SiO₂ | 0.20-0.80 | 0.30-0.60 |

where X is aluminum, gallium, iron, boron, titanium, indium and mixtures thereof, a is 1 or 2, b is 2 when a is 1 (i.e., W is tetravalent); b is 3 when a is 2 (i.e., W is trivalent), M is an alkali metal cation, alkaline earth metal cation or mixtures thereof; n is the valence of M (i.e., 1 or 2); Q is a hexamethylene-1,6-bis-(N-methyl-N-pyrrolidinium) dication and F is fluoride.

As noted above, the SiO₂/XₐO_{b} mole ratio in the reaction mixture is 100 and greater, This means that the SiO₂/XₐO_{b} mole ratio can be infinity, i.e., there is no XₐO_{b} in the reaction mixture. This results in a version of SSZ-74 that is essentially all silica. As used herein, “essentially all silicon oxide” or “essentially all-silica” means that the molecular sieve's crystal structure is comprised of only silicon oxide or is comprised of silicon oxide and only trace amounts of other oxides, such as aluminum oxide, which may be introduced as impurities in the source of silicon oxide.

A preferred source of silicon oxide is tetraethyl orthosilicate. A preferred source of aluminum oxide is LZ-210 zeolite (a type of Y zeolite).

In practice, SSZ-74 is prepared by a process comprising:
(a) preparing an aqueous solution containing (1) a source(s) of silicon oxide, (2) a source(s) of aluminum oxide, gallium oxide, iron oxide, boron oxide, titanium oxide, indium oxide and mixtures thereof, (3) a source of fluoride ion and (4) a hexamethylene-1,6-bis-(N-methyl-N-pyrrolidinium) dication having an anionic counterion which is not detrimental to the formation of SSZ-74;
(b) maintaining the aqueous solution under conditions sufficient to form crystals of SSZ-74; and
(c) recovering the crystals of SSZ-74.

The reaction mixture is maintained at an elevated temperature until the crystals of the SSZ-74 are formed. The hydrothermal crystallization is usually conducted under autogenous pressure, at a temperature between 100° C. and 200° C., preferably between 135° C. and 180° C. The crystallization period is typically greater than 1 day and preferably from about 3 days to about 20 days, The molecular sieve may be prepared using mild stirring or agitation.

During the hydrothermal crystallization step, the SSZ-74 crystals can be allowed to nucleate spontaneously from the reaction mixture. The use of SSZ-74 crystals as seed material can be advantageous in decreasing the time necessary for complete crystallization to occur. In addition, seeding can lead to an increased purity of the product obtained by promoting the nucleation and/or formation of SSZ-74 over any undesired phases. When used as seeds, SSZ74 crystals are added in an amount between 0.1 and 10% of the weight of the first tetravalent element oxide, e.g. silica, used in the reaction mixture.

Once the molecular sieve crystals have formed, the solid product is separated from the reaction mixture by standard mechanical separation techniques such as filtration. The crystals are water-washed and then dried, e.g., at 90° C. to 150° C. for from 8 to 24 hours, to obtain the as-synthesized SSZ-74 crystals. The drying step can be performed at atmospheric pressure or under vacuum.

SSZ-74 as prepared has the X-ray diffraction lines of Table I below. SSZ-74 has a composition, as synthesized (i.e., prior to removal of the SDA from the SSZ-74) and in the anhydrous state, comprising the following (in terms of mole ratios):

| SiO₂/X_{c}O_{d} | greater than 100 |
|---|---|
| M_{2/n}/SiO₂ | 0-0.03 |
| Q/SiO₂ | 0.30-0.70 |
| F/SiO₂ | 0.30-0.70 |

wherein X is aluminum, gallium, iron, boron, titanium, indium and mixtures thereof, c is 1 or 2; d is 2 when c is 1 (i.e., W is tetravalent) or d is 3 or 5 when c is 2 (i.e., d is 3 when W is trivalent or 5 when W is pentavalent), M is an alkali metal cation, alkaline earth metal cation or mixtures thereof; n is the valence of M (i.e., 1 or 2); Q is a hexamethylene-1,6-bis-(N-methyl-N-pyrrolidinium) dication and F is fluoride.

SSZ-74 is characterized by its X-ray diffraction pattern. SSZ-74, as-synthesized, has a crystalline structure whose X-ray powder diffraction pattern exhibits the characteristic lines shown in Table I.

TABLE I
As-Synthesized SSZ-74

| | d-spacing | Relative Integrated |
|---|---|---|
| 2 Theta^{(a)} | (Angstroms) | Intensity (%)^{(b)} |
| 7.95 | 11.11 | W |
| 8.68 | 10.18 | M |
| 8.85 | 9.98 | W-M |
| 9.02 | 9.80 | W |
| 22.69 | 3.92 | W-M |
| 23.14 | 3.84 | VS |
| 24.01 | 3.70 | W |
| 24.52 | 3.63 | W |
| 24.93 | 3.57 | W |
| 29.95 | 2.98 | W |

^{(a)}±0.1
^{(b)}The X-ray patterns provided are based on a relative intensity scale in which the strongest line in the X-ray pattern is assigned a value of 100: W(weak) is less than 20; M(medium) is between 20 and 40; S(strong) is between 40 and 60; VS(very strong) is greater than 60.

Table IA below shows the X-ray powder diffraction lines for as-synthesized SSZ-74 including actual relative intensities.

TABLE IA
As-Synthesized SSZ-74

| 2 Theta^{(a)} | d-spacing (Angstroms) | Intensity |
|---|---|---|
| 7.95 | 11.11 | 7.9 |
| 8.68 | 10.18 | 21.1 |
| 8.85 | 9.98 | 18.7 |
| 9.02 | 9.80 | 11.3 |
| 11.30 | 7.82 | 0.4 |
| 12.70 | 6.96 | 1.8 |
| 13.98 | 6.33 | 2.4 |
| 14.77 | 5.99 | 0.5 |
| 14.85 | 5.96 | 2.1 |
| 15.93 | 5.56 | 6.3 |
| 16.30 | 5.43 | 4.6 |
| 16.50 | 5.37 | 1.8 |
| 17.05 | 5.20 | 0.8 |
| 17.41 | 5.09 | 0.1 |
| 17.71 | 5.00 | 2.0 |
| 18.09 | 4.90 | 7.4 |
| 18.38 | 4.82 | 0.7 |
| 18.89 | 4.69 | 0.9 |
| 18.96 | 4.68 | 4.4 |
| 19.69 | 4.51 | 1.8 |
| 20.39 | 4.35 | 5.1 |
| 20.63 | 4.30 | 4.2 |
| 21.12 | 4.20 | 7.7 |
| 21.55 | 4.12 | 5.4 |
| 21.75 | 4.08 | 0.5 |
| 21.80 | 4.07 | 1.4 |
| 21.88 | 4.06 | 2.1 |
| 21.96 | 4.04 | 1.5 |
| 22.17 | 4.01 | 0.8 |
| 22.69 | 3.92 | 18.9 |
| 23.14 | 3.84 | 100.0 |
| 23.89 | 3.72 | 9.4 |
| 24.01 | 3.70 | 25.6 |
| 24.52 | 3.63 | 13.7 |
| 24.68 | 3.60 | 2.1 |
| 24.93 | 3.57 | 11.3 |
| 25.09 | 3.55 | 0.9 |
| 25.37 | 3.51 | 1.7 |
| 25.57 | 3.48 | 2.7 |
| 26.20 | 3.40 | 5.5 |
| 26.31 | 3.38 | 0.8 |
| 26.67 | 3.34 | 2.0 |
| 26.76 | 3.33 | 1.0 |
| 26.82 | 3.32 | 0.9 |
| 27.01 | 3.30 | 3.4 |
| 27.05 | 3.29 | 0.8 |
| 27.48 | 3.24 | 0.8 |
| 27.99 | 3.19 | 4.2 |
| 28.18 | 3.16 | 0.8 |
| 28.78 | 3.10 | 0.6 |
| 29.03 | 3.07 | 0.7 |
| 29.31 | 3.04 | 0.9 |
| 29.58 | 3.02 | 2.4 |
| 29.95 | 2.98 | 9.6 |
| 30.44 | 2.93 | 3.7 |
| 31.09 | 2.87 | 3.1 |
| 31.36 | 2.85 | 0.8 |
| 31.98 | 2.80 | 2.2 |
| 32.23 | 2.78 | 1.7 |
| 32.37 | 2.76 | 0.6 |
| 32.64 | 2.74 | 1.5 |
| 33.03 | 2.71 | 0.1 |
| 33.34 | 2.69 | 1.0 |
| 33.47 | 2.68 | 1.3 |
| 34.08 | 2.63 | 0.7 |
| 34.55 | 2.59 | 1.8 |
| 34.73 | 2.58 | 0.4 |

^{(a)}±0.1

After calcination, the X-ray powder diffraction pattern for SSZ-74 exhibits the characteristic lines shown in Table II below.

TABLE II
Calcined SSZ-74

| | d-spacing | Relative Integrated |
|---|---|---|
| 2 Theta^{(a)} | (Angstroms) | Intensity (%) |
| 7.98 | 11.07 | M |
| 8.70 | 10.16 | VS |
| 8.89 | 9.93 | S |
| 9.08 | 9.74 | S |
| 14.02 | 6.31 | W |
| 14.93 | 5.93 | M |
| 16.03 | 5.52 | M |
| 23.26 | 3.82 | VS |
| 23.95 | 3.71 | W |
| 24.08 | 3.69 | M |

^{(a)}±0.1

Table IIA below shows the X-ray powder diffraction lines for calcined SSZ-74 including actual relative intensities.

TABLE IIA
Calcined SSZ-74

| | d-spacing | Relative Integrated |
|---|---|---|
| 2 Theta^{(a)} | (Angstroms) | Intensity (%) |
| 7.98 | 11.07 | 34.9 |
| 8.70 | 10.16 | 86.8 |
| 8.89 | 9.93 | 40.2 |
| 9.08 | 9.74 | 47.0 |
| 9.66 | 9.15 | 1.0 |
| 11.26 | 7.85 | 0.4 |
| 11.34 | 7.80 | 0.5 |
| 12.76 | 6.93 | 1.1 |
| 13.26 | 6.67 | 4.6 |
| 14.02 | 6.31 | 13.4 |
| 14.93 | 5.93 | 20.9 |
| 16.03 | 5.52 | 23.5 |
| 16.39 | 5.40 | 4.3 |
| 16.61 | 5.33 | 4.4 |
| 17.12 | 5.18 | 3.0 |
| 17.80 | 4.98 | 2.8 |
| 18.19 | 4.87 | 7.6 |
| 19.05 | 4.66 | 1.9 |
| 19.74 | 4.49 | 0.4 |
| 20.44 | 4.34 | 3.0 |
| 20.75 | 4.28 | 3.4 |
| 21.19 | 4.19 | 7.7 |
| 21.67 | 4.10 | 4.1 |
| 21.99 | 4.04 | 5.8 |
| 22.68 | 3.92 | 3.7 |
| 22.79 | 3.90 | 9.5 |
| 23.26 | 3.82 | 100.0 |
| 23.95 | 3.71 | 14.2 |

^{(a)}±0.1

The X-ray powder diffraction patterns were determined by standard techniques. The radiation was the K-alpha/doublet of copper. The peak heights and the positions, as a function of 2θ where θ is the Bragg angle, were read from the relative intensities of the peaks, and d, the interplanar spacing in Angstroms corresponding to the recorded lines, can be calculated.

The variation in the scattering angle (two theta) measurements, due to instrument error and to differences between individual samples, is estimated at ±0.1 degrees.

Representative peaks from the X-ray diffraction pattern of calcined SSZ-74 are shown in Table II. Calcination can result in changes in the intensities of the peaks as compared to patterns of the “as-made” material, as well as minor shifts in the diffraction pattern.

Crystalline SSZ-74 can be used as-synthesized, but preferably will be thermally treated (calcined). Usually it is desirable to remove the alkali metal cation (if any) by ion exchange and replace it with hydrogen, ammonium, or any desired metal ion.

The original cation in the SSZ-74 can be replaced all or in part by ion exchange with other cations including other metal ions and their amine complexes, alkylammonium ions, ammonium ions, hydrogen ions, and mixtures thereof. Preferred replacing cations are those which render the crystalline SSZ-74 catalytically active. Typical catalytically active ions include hydrogen, metal ions of Groups IB, IIA, IIB, IIIA, VB, VIB and VIII, and of manganese, vanadium, chromium, uranium, and rare earth elements.

Also, water soluble salts of catalytically active materials can be impregnated onto the crystalline SSZ-74. Such catalytically active materials include metals of Groups IB, IIA, IIB, IIIA, IIIB, IVB, VB, VIB, VIIB, and VIII, and rare earth elements.

Ion exchange and impregnation techniques are well known in the art. Typically, an aqueous solution of a cationic species is exchanged one or more times at about 25° C. to about 100° C. A hydrocarbon-soluble metal compound such as a metal carbonyl also can be used to place a catalytically active material. Impregnation of a catalytically active compound on the molecular sieve often results in a suitable catalytic composition. A combination of ion exchange and impregnation can be used. Presence of sodium ion in a composition usually is detrimental to catalytic activity.

The amount of catalytically active material pidaced on the SSZ-74 can vary from about 0.01 weight percent to about 30 weight percent, typically from about 0.05 to about 25 weight percent. The optimum amount can be determined easily by routine experimentation.

SSZ-74 can be formed into a wide variety of physical shapes. Generally speaking, the molecular sieve can be in the form of a powder, a granule, or a molded, product, such as extrudate having a particle size sufficient to pass through a 2-mesh (Tyler) screen and be retained on a 400-mesh (Tyler) screen. In cases where the catalyst is molded, such as by extrusion with an organic binder, the SSZ-74 can be extruded before drying, or, dried or partially dried and then extruded.

SSZ-74 can be composited with other materials resistant to the temperatures and other conditions employed in organic conversion processes. Such matrix materials include active and inactive materials and synthetic or naturally occurring zeolites as well as inorganic materials such as clays, silica and metal oxides. Examples of such materials and the manner in which they can be used are disclosed in U.S. Pat. No. 4,910,006, issued May 20, 1990 to Zones et al., and U.S. Pat. No. 5,316,753, issued May 31, 1994 to Nakagawa, both of which are incorporated by reference herein in their entirety.

### EXAMPLES

The following examples demonstrate but do not limit the present invention.

### Example 1

### Synthesis of Hexamethylene-1,6-bis-(N-methyl-N-pyrrolidinium) dication SDA

In 50 ml of acetone was dissolved 5 ml (48 mmoles) of N-methyl pyrrolidine. 4.9 Grams of 1,6 dibromohexane (20 mmoles) were added and the resulting mixture was stirred at room temperature for three days. Solids formed and were collected by filtration and washed with ether and kept in a vacuum oven. Then 3.71 grams of the dried solid was mixed into 18.7 grams of water and 9.57 grams of AG1-X8 resin for exchange to the OH form. The exchange was run overnight and then the solution was collected and titrated.

### Example 2

Synthesis of All-Silica SSZ-74

6.4 Grams of the solution from Example 1 (3 mmoles) was mixed in a tared Teflon cup with 1.26 grams of tetraethyl orthosilicate and then allowed to evaporate (in a hood) for several days as hydrolysis occurred. A second reaction was set up the same way. After evaporation to the appearance of dryness, one reaction was given 0.20 gram of water and mixed. The second was given 0.60 gram of water and the same treatment ensued. 0.125 Gram of about 50% HF was carefully added to each reaction mixture and the contents were stirred with a plastic spatula and a thick gel formed. In the first case the H2O/SiO2 ratio was now roughly 3.5 and it was 7.0 in the second case. The materials were heated to 150° C. and at 43 RPM in tumbled Parr reactors placed in a Blue M convection heating oven. The reactions were cooled and opened in 6 day periods with a small amount examined by Scanning Electron Microscopy to determine if crystals had formed. After 22 days there was crystalline material in both and the solids were collected (filtration) and washed with copious amounts of water, air dried and then examined by X-ray diffraction (XRD). The product in both cases was SSZ-74.

### Example 3

### Calcination of SSZ-74

The products from both reactions in Example 2 were calcined in stages and in air to 595° C. to remove the organic content. The materials were found to be stable and the XRD patterns showed the relationship to the as-made SSZ-74.

### Example 4

### Adsorption of 2,2-Dimethylbutane

The calcined material of Example 3 was then tested for the uptake of the hydrocarbon 2,2-dimethylbutane. This adsorbate does not enter small pore zeolites (8-ring portals) and sometimes is hindered in entering intermediate pore zeolites like ZSM-5. The SSZ-74 showed a profile more characteristic of intermediate pore materials (as contrasted to Y zeolite, a large pore material), showing steady gradual uptake of the adsorbate.

SSZ-74 was shown to adsorb about 0.08 cc/gram after 3 hours of exposure to the 2,2 dimethyl butane adsorbate using a pulsed mode. This value compares with an analysis for ZSM-5 zeolite which gives a value closer to 0.07 cc/gm at the same point in time under the same experimental conditions. This would indicate that the pores of SSZ-74 are at least 10-rings

### Example 5

### Synthesis of Aluminosilicate SSZ-74

The synthesis parameters of Example 2 were repeated except for the following changes. (1) 0.04 gram of Y zeolite material LZ-210 was added as a potential contributor of Al; (2) the initial H2O/SiO2 ratio for the synthesis was adjusted to 5; (3) seeds of a successful SSZ74 product were added; and (4) the reaction was run at 170° C. After 9 days there was crystalline material which was SSZ-74 when worked up and analyzed by XRD. The solids were calcined then as in Example 3.

### Example 6

### Constraint Index

0.12 grams of the material from Example 5, in a 20-40 pelleted and meshed range, was loaded into a stainless steel reactor and run in a Constraint Index test (50/50 n-hexane/3-methylpentane). The normal feed rate was used (8 μl/min.) and the test was run at 700° F. after the catalyst had been dried in the reactor to near 1000° F. Helium flow was used. At 10 minutes on-stream nearly 30% of the feed was being converted with about equal amounts of each reactant. The selectivity did not change as the catalyst fouled to half the conversion at 100 minutes. The pores of the active SSZ-74 were at least intermediate in size.

### Example 7

### Synthesis of Aluminosilicate SSZ-74

Three mMoles of SDA solution and 1.26 grams (6 mMoles) of tetraethylorthosilicate were combined in a Teflon cup for a Parr reactor. The contents were allowed to react and then most of the water and then the ethanol by-product were allowed to evaporate in a hood over several days. Once the H2O/SiO2 ratio was about 5, from the evaporation, 0.04 grams of LZ-210 zeolite were added (LZ-210 is a Y zeolite which has been treated with (NH₄⁺)₂SiF₆ to provide some de-alumination). A few mg of seeds of SSZ-74 were added in the as-made state. Lastly, 0.132 gram of 50% HF was added and the reactor was closed up and heated at 170° C., 43 RPM, for six days. A sample of the cooled reaction product showed nicely crystalline material in an electron microscope. The reaction contents were worked up and dried. Analysis by X-ray diffraction showed the product to be molecular sieve SSZ-74.

The sample was calcined (in air to 595° C.) and then pelleted and meshed (20-40) and run in a standard Constraint Index test. At 700° F. the initial conversion was 28% with a CI value of 1.1. With time-on-stream the catalyst showed a steady deactivation while the CI value did not change much.

What is claimed is:

1. A process for the preparation of amides from oximes via Beckmann rearrangement comprising contacting the oxime in the vapor phase with a catalyst comprising a crystalline molecular sieve having a mole ratio greater than about 15 of (1) an oxide of a first tetravalent element to (2:) an oxide of a trivalent element, pentavalent element, second tetravalent element which is different: from said first tetravalent element or mixture thereof and having, after calcination, the X-ray diffraction lines of Table II.

2. The process of claim 1 wherein the molecular sieve has a mole ratio greater than about 15 of (1) silicon oxide to (2) an oxide selected from aluminum oxide, gallium oxide, iron oxide, boron oxide, titanium oxide indium oxide and mixtures thereof.

3. The process of claim 1 wherein the oxime is cyclohexanone oxime and the amide is caprolactam.

4. The process of claim 2 wherein the oxime is cyclohexanone oxime and the amide is caprolactam.

5. The process of claim 1 wherein the rearrangement takes place in the presence of a solvent.

6. The process of claim 5 wherein the solvent is of the type R¹—O—R² wherein R¹ is a C₁-C₄ alkyl chain and R² can be a hydrogen atom or an alkyl chain containing a number of carbon atoms less than or equal to R¹.

7. The process of claim 2 wherein the rearrangement takes place in the presence of a solvent.

8. The process of claim 7 wherein the solvent is of the type R¹—O—R² wherein R¹ is a C₁-C₄ alkyl chain and R² can be a hydrogen atom or an alkyl chain containing a number of carbon atoms less than or equal to R¹.
